(19) United States
(12) Patent Application Publication
(10) Pub. No.: US 2022/0062224 A1
(43) Pub. Date: Mar. 3, 2022

(54) SUPPLEMENT THAT ENHANCES INTRACELLULAR CONCENTRATION OF BIOACTIVE MOLECULES THROUGH INHIBITION OF MULTIDRUG RESISTANT (MDR) EFFLUX PUMPS

(71) Applicant: Brilliant Lab LLC, American Fork, UT (US)

(72) Inventors: Daniel A. Gubler, Orem, UT (US); Jeremy Tsang, Vineyard, UT (US)

(21) Appl. No.: 17/407,004

(22) Filed: Aug. 19, 2021

Related U.S. Application Data
(60) Provisional application No. 63/070,080, filed on Aug. 25, 2020.

(51) Int. Cl. A61K 31/352; A61K 31/353; A61K 31/05; A61K 31/192; A61K 31/437; A61K 31/4525; A61K 36/185; A61K 35/644; A61K 31/19; A61K 31/11; A61K 31/09; A61K 31/585; A61K 31/045; A61K 31/722; A61K 36/896

(52) U.S. Cl. CPC A61K 31/352; A61K 36/896; A61K 31/05; A61K 31/192; A61K 31/437; A61K 31/4525; A61K 36/185; A61K 35/644; A61K 31/19; A61K 31/11; A61K 31/09; A61K 31/585; A61K 31/045; A61K 31/722; A61K 31/353

(57) ABSTRACT

Described is a natural product-containing supplement that enhances intracellular concentration of bioactive molecules through inhibition of MDR efflux pumps. Specifically described are dosage forms for administration to a subject, the dosage forms having at least four ingredients selected from the group consisting of apigenin, cannabidiol, chrysin, harmine, caffeic acid, piperine, nobiletin, Hypericum perforatum extract, bee propolis, oleanolic acid, biochanin, luteolin, vanillin, honokiol, pterostilbene, and withaferin A. Upon administration to a mammalian subject, the dosage forms generally inhibit mis-regulated MDR efflux pumps that are responsible for pushing out good nutrients from the cell—thus increasing intracellular concentration of health-promoting compounds.

### PRIORITY CLAIM

This application claims the benefit of the filing date of U.S. Provisional Patent Application Ser. No. 63/070,080, filed Aug. 25, 2020, for “SUPPLEMENT THAT ENHANCES INTRACELLULAR CONCENTRATION OF BIOACTIVE MOLECULES THROUGH INHIBITION OF MULTIDRUG RESISTANT (MDR) EFFLUX PUMPS,” the contents of the entirety of which is incorporated herein by this reference.

### TECHNICAL FIELD

The application relates generally to nutritional supplements and associated methods of making and using them. More particularly, the application relates to a supplement that can enhance the efficacy of other supplements and nutritional food that we eat by inhibiting multidrug resistant (“MDR”) efflux pumps that can remove nutrients from the cell.

### BACKGROUND

The global nutraceutical industry in 2018 was a $231 billion industry with worldwide revenues estimated to reach $336 billion by 2023. Nutraceuticals Global Markets to 2023, (BCC Publishing, July 2018). Nutraceutical formulations have the ability to promote human health in the area of preventative well-care via the science-based model of poly-pharmacology.

Phytonutrient natural products contained in the fruits, vegetables, and other foods that we eat can also promote health. The health food market size was valued at $707 billion in 2016 and is expected to reach $811 billion by 2021. M. Shahbandeh “Health and wellness food market value worldwide 2016-2021,” (Statista, January 2018).

Bioavailability of phytonutrients in both nutritional supplements and good food is a critical component for cellular absorption, clinical efficacy, and the promotion of human health. The ingestion of preventative well-care nutritional supplements and good food is predicated upon the ability of these natural molecules to both enter and remain in the cell so they can exert their biological activity.

Embedded in human cell membranes are multidrug resistant (“MDR”) efflux pumps that are responsible for pumping toxic compounds out of the cell and body, which help to maintain good cellular health. Identified so far in the human cell are over 15 different efflux pumps responsible for pumping out of the cell molecules of varying molecular weights ranging from small molecules to peptides and larger proteins.

Some efflux pumps push small molecules (e.g., phytonutrients) from plants out of the cell. Unfortunately, the selectivity and efficacy of these pumps can be thrown out of balance by poor lifestyle factors (e.g., poor diet, lack of exercise, lack of sleep, too much stress, etc.), causing increased efflux of beneficial bioactive phytonutrients from plants found in nutritional supplements and good food. Excessive efflux of plant bioactive molecules (due to mis-regulated gene expression) can result in lower intracellular concentration of these compounds, and thus reduced overall efficacy of herbal nutritional supplements and healthy food. Efflux pumps that can be mis-regulated and pump out large amounts of health-promoting compounds from the cell include: P-glycoprotein (P-gp), MRP1, MRP2, BCRP, UGT-1A9, and CYP2CA.

### BRIEF SUMMARY

The efficacy of various different nutritional supplements and healthy foods could be improved drastically if natural compounds from plants were able to inhibit mis-regulated MDR efflux pumps with micromolar activity. Described is a nutritional supplement that helps to accomplish this goal and represents an innovative method for increasing the efficacy of both nutritional supplements and good food.

Described herein is a natural product-containing supplement that enhances intracellular concentration of bioactive molecules through inhibition of MDR efflux pumps. Specifically described are novel combinations of natural products from plants having the ability to inhibit a subject's MDR efflux class of pumps that are responsible for pumping bioactive plant phytonutrients out of the cell. The described technology has the ability to enhance the efficacy of natural product-containing nutritional supplements and phytonutrient-laden good foods—thus dramatically improving overall human health.

Examples of readily commercially available nutritional supplements whose efficacy could be enhanced by taking them along with the described supplement(s) include multivitamins, CoQ10, curcumin, traditional Chinese medicine (“TCM”) formulations, ginkgo, vitamin K (“MK7”), alpha-lipoic acid, L-arginine, B-vitamins, adaptogens, probiotics, prebiotics, omega-3 fatty acids, beta-alanine, biotin, SAM, calcium/magnesium, horny goat weed, creatine, maca, and other supplements. In certain embodiments, the described supplement(s) or formulation(s) are taken concurrently with phytonutrient-containing beverages to increase efficacy, such as green tea, black tea, matcha tea, yerba mate, pu-erh tea, guayusa, yaupon, cacao, tejate, and coffee. In certain embodiments, the described supplement(s) or formulation(s) are taken before or concurrently with phytonutrient-containing foods to increase their effects, such as apples, oranges, broccoli, pomegranate, lemons, Brussels sprouts, cacao, lettuce, kale, cauliflower, tomatoes, grapes, cabbage, cranberries, wheat, honey, almonds, walnuts, cashews, beets, carrots, celery, Roquefort cheese and other foods containing large amounts of phytonutrients.

Specifically described herein are dosage forms for administration to a subject, the dosage form comprising at least four ingredients selected from the group consisting of apigenin, cannabidiol, chrysin, harmine, caffeic acid, piperine, nobiletin, Hypericum perforatum extract, bee propolis, oleanolic acid, biochanin, luteolin, vanillin, honokiol, pterostilbene, and withaferin A. In certain embodiments, this dosage form will further include one or more of chitosan, menthol, piperine, or yucca saponins.

In such dosage forms the selected at least four ingredients, when present in a dosage form for once daily administration, are present in the following amounts: from about 25 to about 150 milligrams of apigenin, from about 7 to about 30 milligrams of cannabidiol, from about 180 to about 240 milligrams of chrysin, from about 8 to about 25 milligrams of harmine, from about 75 to about 180 milligrams of caffeic acid, from about 8 to about 30 milligrams of piperine, from about 60 to about 100 milligrams of nobiletin, from about 200 to about 525 milligrams of H. perforatum extract, from about 275 to about 410 milligrams of bee propolis, from about 50 to about 120 milligrams of oleanolic acid, from about 5 to about 30 milligrams of biochanin, from about 20 to about 60 milligrams of luteolin, from about 10 to about 30 milligrams of vanillin, from about 10 to about 40 milligrams of honokiol, from about 180 to about 215 milligrams of pterostilbene, and from about 60 to about 100 milligrams of withaferin a.

In certain preferred embodiments, the dosage form includes from about 140 to about 205 milligrams of chitosan, 5-20 milligrams of piperine, 10-30 milligrams of menthol, and 100-400 milligrams of yucca saponins.

In certain preferred embodiments, the dosage form will include (or be co-administered with) at least one other active ingredient that is pumped out of a subject's cell by at least one efflux pump(s).

Such a dosage form will typically be a softgel, capsule, tablet, gel, powder, gummy, liquid, effervescent, bar, topical patch, serum, lotion, or cream.

Also described are methods of enhancing intracellular concentration of at least one bioactive molecule in a subject, the method comprising administering the described dosage forms to a subject in need thereof.

In certain described methods of enhancing intracellular concentration of at least one bioactive molecule in a subject, the method includes administering a combination of at least four selected ingredients to the subject, wherein the at least four ingredients are selected are from the group consisting of apigenin, cannabidiol, chrysin, harmine, caffeic acid, piperine, nobiletin, Hypericum perforatum extract, bee propolis, oleanolic acid, biochanin, luteolin, vanillin, honokiol, pterostilbene, and withaferin A, wherein administration of the composition regulate at least four efflux pumps of the subject, wherein the efflux pumps are selected from the group consisting of efflux pumps “Breast Cancer Resistance Protein” (or “BCRP”), Cytochrome P4502C8 (“CYP2C8”), MDR-TB, multidrug resistance-associated protein 1 (“MRP1” or “ABCC1”), Multidrug resistance-associated protein 2 (“MRP2,” “canalicular multi specific organic anion transporter 1,” “cMOAT,” ATP-binding cassette sub-family C member 2 (“ABCC2”), P-glycoprotein (P-glycoprotein 1 also known as multidrug resistance protein 1, ATP-binding cassette sub-family B member 1 or “P-gp”), and UGT-1A9 (UDP glucuronosyltransferase family 1 member A9).

In such described methods, the subject ingests the selected at least four ingredients, when selected, in the following amounts on a daily basis: from about 25 to about 150 milligrams of apigenin, from about 7 to about 30 milligrams of cannabidiol, from about 180 to about 240 milligrams of chrysin, from about 8 to about 25 milligrams of harmine, from about 75 to about 180 milligrams of caffeic acid, from about 8 to about 30 milligrams of piperine, from about 60 to about 100 milligrams of nobiletin, from about 200 to about 525 milligrams of Hypericum perforatum extract, from about 275 to about 410 milligrams of bee propolis, from about 50 to about 120 milligrams of oleanolic acid, from about 5 to about 30 milligrams of biochanin, from about 20 to about 60 milligrams of luteolin, from about 10 to about 30 milligrams of vanillin, from about 10 to about 40 milligrams of honokiol, from about 180 to about 215 milligrams of pterostilbene, and from about 60 to about 100 milligrams of withaferin a.

Also described is the dosage form for use in enhancing intracellular concentration of at least one bioactive molecule in a subject.

Further described herein is a method of making such dosage forms, the method comprising admixing the selected ingredients and associating them together into or with the dosage form. The dosage form and method can further include an active or inactive ingredient and/or pharmaceutical excipient into the admixture.

In certain embodiments, the selected phytonutrients may be administered in two different dosage forms, each containing fewer than four of the selected ingredients, but intended to be co-administered to the subject in need thereof so that the at least four ingredients are administered to the subject.

Such dosage forms can be made by admixing the selected phytonutrients and ingredients and associating them together into or with the dosage form.

The dosage form is preferably orally administered to the subject, but other administration modalities are contemplated. When administered orally, the subject ingests the selected at least four phytonutrients.

### DETAILED DESCRIPTION

Phytonutrients are the “medicinal” component of plants and are commonly a starting point in the drug discovery process. The phytonutrients described herein have generally been extracted (e.g., from raw plant/fungal material) and/or standardized to the respective compounds for assimilation into the dosage form(s). Such processing increases reproducibility and quality, reduces the bulk of the selected phytonutrients and allows for smaller, easier to swallow dosage forms.

Although the phytonutrients (and other ingredients) are readily commercially available, processes for extracting phytonutrients are known in the art and may be advantageously utilized herein.

Efflux pumps relevant to this disclosure include ABCG2 (more commonly referred to as Breast Cancer Resistance Protein or “BCRP”), Cytochrome P₄₅₀2C8 (“CYP2C8”), MDR-TB, multidrug resistance-associated protein 1 (“MRP1” or “ABCC1”), Multidrug resistance-associated protein 2 (“MRP2”) also called canalicular multispecific organic anion transporter 1 (“cMOAT”) or ATP-binding cassette sub-family C member 2 (“ABCC2”), P-glycoprotein (P-glycoprotein 1 also known as multidrug resistance protein 1, ATP-binding cassette sub-family B member 1 or “P-gp”), and UGT-1A9 (UDP glucuronosyltransferase family 1 member A9).

Examples of substances that are useful for incorporation in the dosage forms described herein include apigenin, cannabidiol, chrysin, harmine, caffeic acid, piperine, nobiletin, Hypericum perforatum extract, bee propolis, oleanolic acid, biochanin, luteolin, vanillin, honokiol, pterostilbene, and withaferin A. Each such substance is a naturally occurring, albeit purified product (e.g., a flavonoid) that inhibits at least one MDR efflux pump in a subject's cell. Taken together (e.g., administered to a subject who would benefit from such therapy in a non-naturally occurring combination of at least four such substances at dosages and as described herein), the combined substances are believed to act synergistically to inhibit the activity of various MDR efflux pumps in a cell (and at the micromolar level) to reduce ejection of various different beneficial phytonutrients from the treated cell, which joint action greatly enlarges the range of each substance's inherent function, activity, and utility. Such synergistic action reduces ejection of the various bioactive health-promoting compounds from the subject's cells by inhibiting several (e.g., at least four) different MDR efflux pumps as described herein, which pumps would otherwise work together to remove the health-promoting compounds from the cell. Administration of the described dosage forms to a subject thus allows the health-promoting compounds to act beneficially in the cell for a longer period of time, thus treating the subject and/or preventing the occurrence of a malady.

Apigenin is a flavone that is readily commercially available. It has been used as a traditional medicine due to its physiological functions as an antioxidant and anti-inflammatory, its role in lowering blood pressure, and its antibacterial and antiviral properties. Apigenin inhibits the P-gp, BCRP, and MRP2 efflux pumps. As used herein, a typical daily dosage form (e.g., for daily oral administration to an adult human subject) contains from about (plus or minus 5% by weight) 25 to about 150 milligrams of apigenin. The amount can be adjusted for divided doses of the dosage form.

Cannabidiol (“CBD”) is a compound that is readily commercially available. Cannabidiol inhibits the P-gp efflux pump. As used herein, a typical daily dosage form (e.g., for daily oral administration to an adult human subject) contains from about (plus or minus 5% by weight) 7 to about 30 milligrams of cannabidiol. The amount can be adjusted for divided doses of the dosage form.

Chrysin is a flavone that is readily commercially available. It has been used for bodybuilding, treating anxiety, inflammation, gout, HIV/AIDS, preventing cancer, erectile dysfunction (ED), and baldness. It inhibits the BCRP efflux pump. As used herein, a typical daily dosage form (e.g., for daily oral administration to an adult human subject) contains from about (plus or minus 5% by weight) 180 to about 240 milligrams of chrysin. The amount can be adjusted for divided doses of the dosage form.

Harmine is a compound that is readily commercially available. It has been used for treating diabetes. It inhibits the BCRP efflux pump. As used herein, a typical daily dosage form (e.g., for daily oral administration to an adult human subject) contains from about (plus or minus 5% by weight) 8 to about 25 milligrams of harmine. The amount can be adjusted for divided doses of the dosage form.

Caffeic acid is a compound that is readily commercially available. It inhibits the P-gp, MRP1, MRP2, and BCRP efflux pumps. It is used in supplements for boosting athletic performance, exercise-related fatigue, weight loss, cancer, HIV/AIDS, and herpes. As used herein, a typical daily dosage form (e.g., for daily oral administration to an adult human subject) contains from about (plus or minus 5% by weight) 75 to about 180 milligrams of caffeic acid. The amount can be adjusted for divided doses of the dosage form.

Piperine is a compound that is readily commercially available. It has been shown to help relieve nausea, headaches, and poor digestion and has anti-inflammatory properties. It inhibits the P-gp, CYP2C8, BCRP, MRP1, and MRP2 efflux pumps. As used herein, a typical daily dosage form (e.g., for daily oral administration to an adult human subject) contains from about (plus or minus 5% by weight) 8 to about 30 milligrams of piperine. The amount can be adjusted for divided doses of the dosage form.

Nobiletin is a flavonoid that is readily commercially available. It has been used to rescue bulbectomy-induced memory impairment. It inhibits the P-gp, and MRP1 efflux pumps. As used herein, a typical daily dosage form (e.g., for daily oral administration to an adult human subject) contains from about (plus or minus 5% by weight) 60 to about 110 milligrams of nobiletin. The amount can be adjusted for divided doses of the dosage form.

Hypericum perforatum extract (“St. John's Wort”) that is readily commercially available. The extract is known to have antidepressant properties and has been used to treat mild to moderate depression, anxiety, and sleep disorders. It inhibits the P-gp, and MRP1 efflux pumps. As used herein, a typical daily dosage form (e.g., for daily oral administration to an adult human subject) contains from about (plus or minus 5% by weight) 200 to about 525 milligrams of H. perforatum extract. The amount can be adjusted for divided doses of the dosage form.

Bee propolis is readily commercially available. It inhibits the P-gp, MRP1, MRP2, and BCRP efflux pumps. It is used to treat diabetes, cold sores, swelling (inflammation), and sores inside the mouth (oral mucositis). As used herein, a typical daily dosage form (e.g., for daily oral administration to an adult human subject) contains from about (plus or minus 5% by weight) 275 to about 410 milligrams of bee propolis. The amount can be adjusted for divided doses of the dosage form.

Oleanolic acid is a compound that is readily commercially available. It exhibits antitumor and antiviral properties. It inhibits the MRP1 efflux pump. As used herein, a typical daily dosage form (e.g., for daily oral administration to an adult human subject) contains from about (plus or minus 5% by weight) 50 to about 120 milligrams of oleanolic acid. The amount can be adjusted for divided doses of the dosage form.

Biochanin A is an O-methylated isoflavone that is readily commercially available. It exhibits antitumor and cardiovascular properties. It inhibits the P-gp efflux pump. As used herein, a typical daily dosage form (e.g., for daily oral administration to an adult human subject) contains from about (plus or minus 5% by weight) 5 to about 30 milligrams of biochanin. The amount can be adjusted for divided doses of the dosage form.

Luteolin is a flavone that is readily commercially available. It has been used for treating hypertension, inflammatory disorders, and cancer. It inhibits the P-gp, and MRP2 efflux pumps. As used herein, a typical daily dosage form (e.g., for daily oral administration to an adult human subject) contains from about (plus or minus 5% by weight) 20 to about 60 milligrams of luteolin. The amount can be adjusted for divided doses of the dosage form.

Vanillin is a compound that is readily commercially available. It is the primary component of the extract of vanilla bean. It inhibits the MDR-TB efflux pump. As used herein, a typical daily dosage form (e.g., for daily oral administration to an adult human subject) contains from about (plus or minus 5% by weight) 10 to about 30 milligrams of vanillin. The amount can be adjusted for divided doses of the dosage form.

Honokiol is a lignin compound and is readily commercially available. It inhibits the P-gp, MRP1, MRP2, and BCRP efflux pumps. It has been identified as having therapeutic potential in anxiety, pain, cerebrovascular injury, epilepsy, and cognitive disorders including Alzheimer's disease. As used herein, a typical daily dosage form (e.g., for daily oral administration to an adult human subject) contains from about (plus or minus 5% by weight) 10 to about 40 milligrams of honokiol. The amount can be adjusted for divided doses of the dosage form.

Pterostilbene is a compound that is readily commercially available. It exhibits antioxidant, anti-inflammatory, and anti-carcinogenic properties leading to improved function of normal cells and inhibition of malignant cells. It inhibits the UGT-1A9 efflux pump. As used herein, a typical daily dosage form (e.g., for daily oral administration to an adult human subject) contains from about (plus or minus 5% by weight) 180 to about 215 milligrams of pterostilbene. The amount can be adjusted for divided doses of the dosage form.

Withaferin A is a compound that is readily commercially available. Withaferin A is a steroidal lactone, derived from Acnistus arborescens or Withania somnifera (Indian Winter cherry). W. somnifera, commonly called “ashwagandha,” is used to treat asthma, diabetes, hypertension, stress, arthritic diseases, and cancer. It inhibits the MDR-TB efflux pump. As used herein, a typical daily dosage form (e.g., for daily oral administration to an adult human subject) contains from about (plus or minus 5% by weight) 60 to about 100 milligrams of withaferin A. The amount can be adjusted for divided doses of the dosage form.

Preferably, one or more of chitosan (typically at a daily dosage of from about 140 to about 205 mg), menthol (typically at a daily dosage range of from about 5 to about 20 milligrams), piperine (typically at a daily dosage range of from about 10 to about 30 milligrams), or yucca saponins (typically at a daily dosage range of from about 100 to about 400 milligrams), all of which are readily commercially available, are included in the formulation as adjuvant(s) to increase both bioavailability and small molecule permeability.

In certain embodiments, other organic or inorganic bioactive ingredients can be included in the formulation in order to enhance efficacy.

Once being apprised of the instant disclosure, a person of ordinary skill in the art will be readily able to make or prepare the dosage forms using Galenical techniques. Preferably, a finished product delivery forms is selected from the group consisting of a softgel, capsule, tablet, gel, powder, gummy, liquid, effervescent, bar, topical patch, serum, lotion, and cream.

The described composition is useful as a nutritional supplement capable of increasing the intracellular concentration and efficacy of health-promoting phytonutrients by inhibiting efflux pumps at micromolar amounts, which would otherwise pump them out of the cell.

The invention is further described with the aid of the following illustrative Examples.

### Example I

The following phytonutrients are thoroughly admixed as close to uniform consistency as possible:

| | | |
|---|---|---|
| Apigenin | 80 | g |
| Cannabidiol | 20 | g |
| Chrysin | 200 | g |
| Caffeic acid | 125 | g |
| Piperine | 20 | g |
| Chitosan | 150 | g |

The resulting admixture is divided into 1,000 equal portions each placed into one of 1,000 appropriately-sized hard (or vegan) gelatin capsules with or without a pharmaceutically acceptable diluent.

### Example II

Capsules of EXAMPLE I are administered in a dosing regimen of one capsule daily (e.g., with a nutritious lunch and green tea) to a subject. The treatment regimen is continued for 13 weeks. The described supplement supports the subject's health via, for example, inhibiting efflux pumps.

### Example III

The following phytonutrients are thoroughly admixed as close to uniform consistency as possible:

| | | |
|---|---|---|
| Cannabidiol | 20 | g |
| Harmine | 20 | g |
| Caffeic acid | 100 | g |
| Piperine | 20 | g |
| Chitosan | 150 | g |
| Yucca saponins | 325 | g |
| Withaferin a | 100 | g |

The resulting admixture is divided into 1,000 equal portions each placed into one of 1,000 appropriately-sized hard (or vegan) gelatin capsules with or without a pharmaceutically acceptable diluent.

### Example IV

Capsules of EXAMPLE III are administered in a dosing regimen of one capsule daily to a subject undergoing pharmacological treatment for tuberculosis. The treatment regimen is continued for 6 months. The described supplement supports the subject's health via, for example, assisting in the treatment of tuberculosis. The dosage of the pharmacological treatment for tuberculosis co-administered to the subject may need to be adjusted (e.g., reduced) by the subject's health care provider.

### Example V

The following phytonutrients are thoroughly admixed as close to uniform consistency as possible:

| | | |
|---|---|---|
| Cannabidiol | 20 | g |
| Bee Propolis | 350 | g |
| Chrysin | 40 | g |
| Piperine | 20 | g |
| Chitosan | 190 | g |
| Luteolin | 40 | g |
| Trans-Pterostilbene | 175 | g |

The resulting admixture is divided into 1,000 equal portions each placed into one of 1,000 appropriately-sized hard (or vegan) gelatin capsules with or without a pharmaceutically acceptable diluent.

### Example VI

Capsules of EXAMPLE V are administered in a dosing regimen of one capsule daily (with a dinner of non-junk food by increasing intracellular concentration of the health-promoting compounds found in the good food dinner) to a subject in order to enhance health. The treatment regimen is continued for 26 weeks.

### Example VII

The following phytonutrients are thoroughly admixed as close to uniform consistency as possible:

| | | |
|---|---|---|
| Nobiletin | 100 | g |
| Hypericum perforatum extract | 350 | g |
| Oleanolic acid | 85 | g |
| Biochanin | 20 | g |
| Chitosan | 190 | g |
| Vanillin | 20 | g |
| Honokiol | 25 | g |

The resulting admixture is divided into 1,000 equal portions each placed into one of 1,000 appropriately-sized hard (or vegan) gelatin capsules with or without a pharmaceutically acceptable diluent.

### Example VIII

Capsules of EXAMPLE VII are administered in a dosing regimen of one capsule daily (with a cup of tea) to a subject in order to enhance health. The treatment regimen is continued for a year.

### REFERENCES

(The contents of each of which are incorporated herein by this reference.)
Albassam, A. A. & Frye, R. F. Effect of pterostilbene on in vitro drug metabolizing enzyme activity. Saudi Pharm J 27, 406-412 (2019).
Ali, A. & Ahmed, S. A review on chitosan and its nanocomposites in drug delivery. Int J Biol Macromol 109, 273-286 (2017).
Ampasavate, C., Sotanaphun, U., Phattanawasin, P. & Piyapolrungroj, N. Effects of Curcuma spp. on P-glycoprotein function. Phytomedicine Int J Phytotherapy Phytopharm 17, 506-12 (2009).
An, G., Gallegos, J. & Morris, M. E. The bioflavonoid kaempferol is an Abcg2 substrate and inhibits Abcg2-mediated quercetin efflux. Drug Metabolism Dispos Biological Fate Chem 39, 426-32 (2010).
Assa, F. et al. Chitosan magnetic nanoparticles for drug delivery systems. Crit Rev Biotechnol 37, 492-509 (2016).
Bi, X. et al. Piperine enhances the bioavailability of silybin via inhibition of efflux transporters BCRP and MRP2. Phytomedicine Int J Phytotherapy Phytopharm 54, 98-108 (2018).
Chen, L., Long, X., Huang, S., Wu, H. & Pan, S. [Drug delivery systems of baicalin, baicalin-phospholipid complex and self-microemulsifying drug across Caco-2 cell model]. Zhong Yao Cai Zhongyaocai J Chin Medicinal Mater 35, 757-61 (2012).
Dellinger, R. W., Garcia, A. M. G. & Meyskens, F. L. Differences in the Glucuronidation of Resveratrol and Pterostilbene: Altered Enzyme Specificity and Potential Gender Differences. Drug Metab Pharmacok 29, 112-119 (2014).
Ferreira, A., Rodrigues, M., Fortuna, A., Falcão, A. & Alves, G. Flavonoid compounds as reversing agents of the P-glycoprotein-mediated multidrug resistance: An in vitro evaluation with focus on antiepileptic drugs. Food Res Int 103, 110-120 (2018).
Fleisher, B., Unum, J., Shao, J. & An, G. Ingredients in Fruit Juices Interact with Dasatinib Through Inhibition of BCRP: A New Mechanism of Beverage-Drug Interaction. J Pharm Sci 104, 266-275 (2015).
Gerber, W. et al. Capsaicin and Piperine as Functional Excipients for Improved Drug Delivery across Nasal Epithelial Models. Planta Med 85, 1114-1123 (2019).
Gou, J. et al. Absorption properties and effects of caffeic acid phenethyl ester and its p-nitro-derivative on P-glycoprotein in Caco-2 cells and rats. Pharm Biol 54, 1-8 (2016).
He, X.-W. et al. Anti-atherosclerotic potential of baicalin mediated by promoting cholesterol efflux from macrophages via the PPARγ-LXRα-ABCA1/ABCG1 pathway. Biomed Pharmacother Biomedecine Pharmacother 83, 257-264 (2016).
Jiang, L. et al. Pterostilbene supplements carry the risk of drug interaction via inhibition of UDP-glucuronosyltransferases (UGT) 1A9 enzymes. Toxicol Lett 320, 46-51 (2020).
Kalapos-Kovacs, B. et al. Multiple ABC Transporters Efflux Baicalin. Phytotherapy Res Ptr 29, 1987-90 (2015).
Kaur, M. & Badhan, R. K. S. Phytochemical mediated-modulation of the expression and transporter function of breast cancer resistance protein at the blood-brain barrier: An in-vitro study. Brain Res 1654, 9-23 (2017).
Kesarwani, K., Gupta, R. & Mukerjee, A. Bioavailability enhancers of herbal origin: an overview. Asian Pac J Tropical Biomed 3, 253-66 (2013).
Khdair, A. et al. Modified-chitosan nanoparticles: Novel drug delivery systems improve oral bioavailability of doxorubicin. European J Pharm Sci Official J European Fed Pharm Sci 93, 38-44 (2016).
Kotwal, P. et al. Effect of Natural Phenolics on Pharmacokinetic Modulation of Bedaquiline in Rat to Assess the Likelihood of Potential Food-Drug Interaction. J Agr Food Chem 68, 1257-1265 (2020).
Kumar, A. & Jaitak, V. Natural products as multidrug resistance modulators in cancer. Eur J Med Chem 176, 268-291 (2019).
Lee, S. H., Lee, Y.-S., Song, J. G. & Han, H.-K. Improved In vivo Effect of Chrysin as an Absorption Enhancer Via the Preparation of Ternary Solid Dispersion with Brij® L4 and Aminoclay. Curr Drug Deliv 16, 86-92 (2019).
Li, S. et al. Piperine, a piperidine alkaloid from Piper nigrum re-sensitizes P-gp, MRP1 and BCRP dependent multidrug resistant cancer cells. Phytomedicine 19, 83-87 (2011).
Mohana, S. et al. Screening dietary flavonoids for the reversal of P-glycoprotein-mediated multidrug resistance in cancer. Mol Biosyst 12, 2458-70 (2016).
Mohos, V. et al. Effects of chrysin and its major conjugated metabolites chrysin-7-sulfate and chrysin-7-glucuronide on cytochrome P450 enzymes, and on OATP, P-gp, BCRP and MRP2 transporters. Drug Metabolism Dispos Biological Fate Chem DMD-AR-2020-000085 (2020) doi:10.1124/dmd.120.000085.
Moussaoui, S. & Lahouel, M. Propolis Extract: A Potent Bacteria Efflux Pump Inhibitor. J Biologically Active Prod Nat 4, 216-223 (2014).
Muthusamy, G. et al. Ferulic acid reverses ABCB1-mediated paclitaxel resistance in MDR cell lines. Eur J Pharmacol 786, 194-203 (2016).
Nabekura, T., Yamaki, T. & Kitagawa, S. Effects of chemopreventive citrus phytochemicals on human P-glycoprotein and multidrug resistance protein 1. Eur J Pharmacol 600, 45-49 (2008).
Nabekura, T., Kawasaki, T., Furuta, M., Kaneko, T. & Uwai, Y. Effects of Natural Polyphenols on the Expression of Drug Efflux Transporter P-Glycoprotein in Human Intestinal Cells. Acs Omega 3, 1621-1626 (2018).
Prasch, S. et al. Resistance modulatory and efflux-inhibitory activities of capsaicinoids and capsinoids. Bioorg Chem 82, 378-384 (2019).
Takara, K. et al. Effects of propolis extract on sensitivity to chemotherapeutic agents in HeLa and resistant sublines. Phytother Res 21, 841-846 (2007).
Tan, K. W., Li, Y., Paxton, J. W., Birch, N. P. & Scheepens, A. Identification of novel dietary phytochemicals inhibiting the efflux transporter breast cancer resistance protein (BCRP/ABCG2). Food Chem 138, 2267-2274 (2013).
Teng, Y.-N., Wang, C. C. N., Liao, W.-C., Lan, Y.-H. & Hung, C.-C. Caffeic Acid Attenuates Multi-Drug Resistance in Cancer Cells by Inhibiting Efflux Function of Human P-glycoprotein. Mol Basel Switz 25, 247 (2020).
Xie, Y., Zhang, D., Zhang, J. & Yuan, J. Metabolism, Transport and Drug-Drug Interactions of Silymarin. Molecules 24, 3693 (2019).

What is claimed is:

1. A dosage form for administration to a subject, the dosage form comprising at least four ingredients selected from the group consisting of apigenin, cannabidiol, chrysin, harmine, caffeic acid, piperine, nobiletin, Hypericum perforatum extract, bee propolis, oleanolic acid, biochanin, luteolin, vanillin, honokiol, pterostilbene, and withaferin A.

2. The dosage form of claim 1, wherein the dosage form further comprises one or more of chitosan, menthol, piperine, or yucca saponins.

3. The dosage form of claim 2, wherein the dosage form comprises from about 140 to about 205 milligrams of chitosan, from about 5 to about 30 milligrams of menthol, and/or from about 100 to about 400 milligrams of yucca saponins.

4. The dosage form of claim 1, wherein the selected at least four ingredients, when present, are present in the following amounts:
from about 25 to about 150 milligrams of apigenin,
from about 7 to about 30 milligrams of cannabidiol,
from about 180 to about 240 milligrams of chrysin,
from about 8 to about 25 milligrams of harmine,
from about 75 to about 180 milligrams of caffeic acid,
from about 8 to about 30 milligrams of piperine,
from about 60 to about 100 milligrams of nobiletin,
from about 200 to about 525 milligrams of Hypericum perforatum extract,
from about 275 to about 410 milligrams of bee propolis,
from about 50 to about 120 milligrams of oleanolic acid,
from about 5 to about 30 milligrams of biochanin,
from about 20 to about 60 milligrams of luteolin,
from about 10 to about 30 milligrams of vanillin,
from about 10 to about 40 milligrams of honokiol,
from about 180 to about 215 milligrams of pterostilbene, and
from about 60 to about 100 milligrams of withaferin a.

5. The dosage form of claim 1, wherein the dosage form is selected from the group consisting of a softgel, capsule, tablet, gel, powder, gummy, liquid, effervescent, bar, topical patch, serum, lotion, and cream.

6. The dosage form of claim 1, further comprising:
at least one other active ingredient that is pumped out of a subject's cell by at least one efflux pump(s).

7. The dosage form of claim 1, comprising: cannabidiol, bee propolis, chrysin, piperine, chitosan, luteolin, and trans-pterostilbene.

8. A method of enhancing intracellular concentration of at least one bioactive molecule in a subject, the method comprising:
administering the dosage form of claim 1 to the subject.

9. A method of enhancing intracellular concentration of at least one bioactive molecule in a subject, the method comprising:
administering a combination of at least four selected ingredients to the subject, wherein the at least four ingredients are selected are from the group consisting of apigenin, cannabidiol, chrysin, harmine, caffeic acid, piperine, nobiletin, Hypericum perforatum extract, bee propolis, oleanolic acid, biochanin, luteolin, vanillin, honokiol, pterostilbene, and withaferin A,
wherein administration of the composition regulate at least four efflux pumps of the subject, wherein the efflux pumps are selected from the group consisting of efflux pumps ABCG2 (more commonly referred to as Breast Cancer Resistance Protein or “BCRP”), Cytochrome P₄₅₀2C8 (“CYP2C8”), MDR-TB, multidrug resistance-associated protein 1 (“MRP1” or “ABCC1”), Multidrug resistance-associated protein 2 (“MRP2,” “canalicular multispecific organic anion transporter 1,” “cMOAT,” ATP-binding cassette sub-family C member 2 (“ABCC2”), P-glycoprotein (P-glycoprotein 1 also known as multidrug resistance protein 1, ATP-binding cassette sub-family B member 1 or “P-gp”), and UGT-1A9 (UDP glucuronosyltransferase family 1 member A9).

10. The method according to claim 9, wherein the subject ingests the selected at least four ingredients, when selected, in the following amounts on a daily basis:
from about 25 to about 150 milligrams of apigenin,
from about 7 to about 30 milligrams of cannabidiol,
from about 180 to about 240 milligrams of chrysin,
from about 8 to about 25 milligrams of harmine,
from about 75 to about 180 milligrams of caffeic acid,
from about 8 to about 30 milligrams of piperine,
from about 60 to about 100 milligrams of nobiletin,
from about 200 to about 525 milligrams of Hypericum perforatum extract,
from about 275 to about 410 milligrams of bee propolis,
from about 50 to about 120 milligrams of oleanolic acid,
from about 5 to about 30 milligrams of biochanin,
from about 20 to about 60 milligrams of luteolin,
from about 10 to about 30 milligrams of vanillin,
from about 10 to about 40 milligrams of honokiol,
from about 180 to about 215 milligrams of pterostilbene, and
from about 60 to about 100 milligrams of withaferin a.

11. A method of enhancing intracellular concentration of at least one bioactive molecule in a subject, the method comprising:
administering the dosage form of claim 1 to the subject so as to enhance intracellular concentration of the at least one bioactive molecule in the subject.

12. A method of making the dosage form of claim 1, the method comprising:
admixing the selected ingredients to form an admixture, and
associating the admixture into or with the dosage form.

13. The method according to claim 12, further comprising including an inactive ingredient and/or at least one pharmaceutical excipient into the admixture.
